(19) United States
(12) Patent Application Publication
(10) Pub. No.: US 2007/0249029 A1
(43) Pub. Date: Oct. 25, 2007

(54) Self-Sustaining and Continuous System and Method of Anaerobically Digesting Ethanol Stillage

(76) Inventors: Richard M. Marshall, Lake Country (CA); Alexander V. Kopp, Lake Country (CA)

Correspondence Address: ANTONY C. EDWARDS, SUITE 200 - 270 HIGHWAY 33 WEST, KELOWNA, BC, V1X 1X7, CA

(21) Appl. No.: 11/660,699

(22) PCT Filed: Aug. 23, 2005
(86) PCT No.: PCT/CA05/01284

§ 371 Date: Feb. 21, 2007

Related U.S. Application Data
(60) Provisional application No. 60/603,279, filed on Aug. 23, 2004.

(51) Int. Cl. C12P 7/06; C12C 1/15

(52) U.S. Cl. 435/161; 435/291.1

(57) ABSTRACT

A system and method of anaerobically digesting ethanol stillage and reintegrating substantially all by-products thereof back into the system is disclosed. The system includes an ethanol producing facility for producing ethanol and an anaerobic digestion facility for anaerobically digesting stillage from the ethanol producing facility to produce a plurality of by-products. A plurality of sub-systems utilize the plurality of by-products from anaerobic digestion to produce a plurality of end-products. At least one of the plurality of end-products from the various sub-systems is integrated back into the ethanol producing facility and into at least one of the sub-systems such that the system of anaerobically digesting stillage is a continuous and self-sustaining operation.

### FIELD OF THE INVENTION

The present invention relates to the field of anaerobic digestion, and more particularly, it relates to a system of anaerobically digesting distillery stillage and using the by-products thereof within an integrated process.

### BACKGROUND OF THE INVENTION

Ethanol is an alcohol made by fermenting and distilling simple sugars. Typically, ethanol is produced from crops such as corn, grain, wheat, sugar, and other agricultural feedstocks. Zymase, or other enzymes from yeast, changes the crops into simple sugars after they have been ground and slurried with water. The fermentation reaction converts the simple sugars into ethanol and carbon dioxide. The ethanol is then concentrated by distillation such that the composition of the vapor from aqueous ethanol is 96 percent ethanol and 4 percent water. Dehydrating agents may be used to remove the remaining water to produce absolute ethanol. Because ethanol is produced from crops or plants that harness the power of the sun, it is considered a renewable fuel.

Ethanol is miscible and therefore useful as a solvent for many substances and in making perfumes, paints, lacquers, and explosives. Ethanol may also be added to gasoline to form cleaner burning fuel. Gasoline comprises many toxic chemicals such as benzene. By adding ethanol, which contains 35% oxygen, the potency of the toxic chemicals in gasoline is diluted. Because ethanol molecules contain oxygen, ethanol added gasoline burns more completely, which results in fewer emissions and helps reduce air pollution.

Whole stillage is the residual by-product from the distillation of ethanol. Up to 20 litres of whole stillage may be generated for each litre of ethanol produced. Whole stillage is typically separated by centrifugation into a coarse grain fraction called wet cake and an aqueous fraction called thin stillage. Conventionally, the wet cake and thin stillage are dried by evaporation and natural gas dryers and the remaining solids are sold as animal feed. Whole stillage may have a considerable pollution potential that exceeds a chemical oxygen demand (COD) of 100 g/L, depending on the production process and the feedstock used. For example, the use of molasses as feedstock is associated with high levels of sulphates in the stillage and barley fermentation produces stillage having high nitrogen content. Furthermore, heavy metals such as copper, chromium, nickel and zinc may also be found in the effluent due to corrosion ofpiping, tanks, and heat exchangers.

The problem with processing whole stillage in the above described manner is the high capital and operation costs and energy demand associated with separating, evaporating, and treating the whole stillage. Up to 50% of the cost of operating an ethanol facility is devoted to drying whole stillage constituents by separation and evaporation and processing the effluent in a manner such that environmental standards are met. To reduce such costs, anaerobic treatment of whole or thin stillage has been developed as an effective and economic treatment option.

Anaerobic digestion is a biological process that produces biogases such as methane and carbon dioxide from organic wastes. The advantage of anaerobic digestion is that it reduces odor and water pollution caused by unprocessed wastes and produces a biogas fuel that can be used for process heating and/or electricity generation.

Anaerobic digestion typically occurs in an airtight container called a digester. The process of anaerobic digestion consists of three steps. First, the organic matter is decomposed to break down the organic material to usable-sized molecules such as sugar. The second step converts the decomposed matter to organic acids. Finally, the acids are converted to methane gas and carbon dioxide. Depending on the waste feedstock and the system design, biogas is typically 55 to 75 percent pure methane. The collected methane may fuel an engine-generator to generate electricity.

The problem with current systems of anaerobically treating ethanol stillage is that various other by-products of anaerobic digestion are wasted or are not fully utilized. Furthermore, conventionally, the lack of synergies, that is efficient cooperation, between the anaerobic digestion facility and other systems discourage commercial usage because the cost of the overall system cannot be economically justified. U.S. Pat. No. 6,355,456 to Hallberg et al. discloses a system wherein a feed yard, an anaerobic digestion system, and an ethanol plant are integrated in a continuous operation to create what is disclosed as a cost-effective system and environmentally friendly livestock feeding operation. Hallberg describes the use of ethanol stillage as feed for livestock in the feed yard, anaerobically digesting the manure from the livestock to produce methane, and converting the methane into electricity to operate the ethanol plant. Although Hallberg discloses a synergistic system, it fails to provide a fully self-contained and self-sustaining system whereby all by-products of the anaerobically treated organic material are fully re-integrated into the system. In addition the cattle eat the byproduct therefore making more waste product and CO₂.

Therefore, there is a need for a synergistic system of anaerobically treating ethanol stillage wherein all or substantially all of the by-products thereof are used and re-integrated back into the system such that the system is a continuous and autonomous operation.

### SUMMARY OF THE INVENTION

An object of the present invention is to provide a synergistic system whereby whole or thin stillage from an ethanol facility is anaerobically digested and the by-products thereof may be used by various other sub-systems.

Another object of the invention is to provide an integrated, self-sufficient system such that the synergistic interactions between each sub-system taken together create an economically viable operation of each of the various sub-systems.

Another object of the invention is to provide an ethanol facility, an anaerobic digestion facility for digesting ethanol stillage, a greenhouse, a generator, and an ethanol user such that each of the subsystems is integrated with one another to form a self-sustaining and independent unit.

Another object of the invention is to provide an integrated system that is environmentally friendly by recycling and/or using virtually all by-products of each system, therefore making the ethanol production an environmentally neutral or positive net process.

The present invention provides a synergistic system of anaerobically digesting ethanol stillage and reintegrating substantially all by-products thereof back into the system. The system includes an ethanol producing facility for producing ethanol and an anaerobic digestion facility for anaerobically digesting stillage from the ethanol producing facility to produce a plurality of by-products. A plurality of sub-systems utilize the plurality of by-products from anaerobic digestion to produce a plurality of end-products. At least one of the plurality of end-products from the various sub-systems is integrated back into the ethanol producing facility and into at least one of the sub-systems such that the system of anaerobically digesting stillage is a continuous and self-sustaining operation.

The plurality of sub-systems include a generator sub-system for producing electricity, a greenhouse sub-system for producing greenhouse end-products, and an ethanol user sub-system for producing ethanol end-products and an organic fertilizer subsystem. Each of the sub-systems, the ethanol producing facility, and the anaerobic digestion facility are locatable within close proximity to one another such that the plurality of sub-systems, the ethanol producing facility, and the anaerobic digestion facility, taken together, form a self-contained tightly integrated unit. The ethanol end-products produced by ethanol users include herbal remedies and tinctures, fuel oxygenate, fuel additive, and industrial solvents.

The ethanol producing facility further produces carbon dioxide which may be transported to the greenhouse sub-system such that the carbon dioxide may facilitate photosynthesis of the biomass and other greenhouse end-products. The greenhouse end-products also include a plurality of herbs and plants that may be supplied to the natural products manufacturer for producing herbal remedies and tinctures with the ethanol. Waste from the greenhouse sub-system may be added to the whole stillage for anaerobic digestion at the anaerobic digestion facility.

Stillage from the ethanol producing facility is transported to the anaerobic digestion facility which is substantially adjacent to the ethanol producing facility. In addition to the stillage, any organic waste and any organic discard from the plurality of sub-systems may be added to the stillage to be anaerobically digested at the anaerobic digestion facility. The anaerobic digestion facility comprises at least one air tight digester for receiving the stillage, the organic waste, and the organic discard for anaerobic digestion. The plurality of by-products produced from the anaerobic digestion facility include methane gas, carbon dioxide, hot water, and effluent. The digester is a continuous digester wherein the stillage, the organic waste, and the organic discard are continually fed into the digester such that methane gas and carbon dioxide are continually produced, and the effluent and the hot water are continually removed from the digester.

The methane gas may be collected from the digester and scrubbed and compressed to be supplied as natural gas to various consumers. Alternatively, the methane gas may be compressed and provided as a natural gas supply. Alternatively, the methane gas may be transported from the digester to the generator sub-system to produce electricity. The generator sub-system comprises a boiler wherein the methane gas is burned to heat the boiler. Steam produced by the boiler drives a turbine which turns electric generators to produce electricity. The electricity may be sold to a utilities company via a substation or the electricity may be integrated back into at least one of the plurality of sub-systems to operate the sub-system. Preferably, the electricity is integrated back into the ethanol producing facility to operate the ethanol producing facility. Heat and steam produced from converting the methane gas to electricity may be collected and transported to the ethanol producing facility and used to aid in the fermentation and distillation process.

Carbon dioxide produced in the digester may be collected and transported to said greenhouse sub-system to facilitate photosynthesis of the greenhouse end products. Hot water from the digester may be collected and used for heating the greenhouse facility of the greenhouse sub-system. Effluent from the digester may be collected and used as an organic and pathogen free soil conditioner to facilitate growth of the greenhouse end-products. Alternatively, the effluent may be separated into a solid and a liquid wherein the solid may be used as compost and the liquid used as a fertilizer to facilitate growth of the greenhouse end-products. The remaining liquid may be subjected to reverse osmosis to create purified water. The purified water may be reintroduced into the ethanol producing facility to produce ethanol.

### BRIEF DESCRIPTION OF THE DRAWINGS

FIG. 1 is a flow chart depicting a system of anaerobically digesting ethanol stillage and using by-products thereof according to one embodiment of the present invention.

### DETAILED DESCRIPTION OF PREFERRED EMBODIMENTS

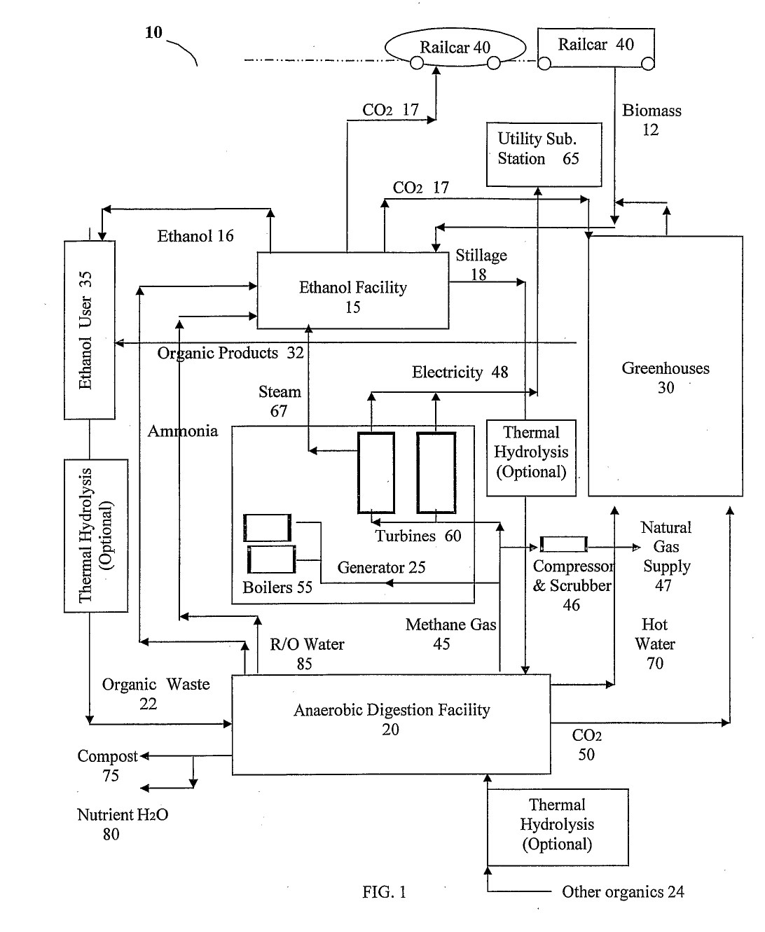

FIG. 1 depicts a synergistic system of anaerobically digesting ethanol stillage and using by-products thereof, the system 10 comprising an ethanol producing facility 15, an anaerobic digestion facility 20, a generator 25, a greenhouse 30, and an ethanol user 35.

To synthesize ethanol, biomass 12 such as sugar crops (i.e. sugar cane, sugar beets), starch crops (i.e. corn, grain, wheat), or cellulosic materials (i.e. crop residues, municipal solid waste, wood) are transported via railcar 40 or any other form of transportation from various sources and milled or otherwise broken-down and prepared for fermentation and distillation in ethanol producing facility 15. Greenhouse 30 may also produce and supply the necessary biomass, in part or in whole, for the production of ethanol at ethanol producing facility 15. Generally, the process of producing ethanol typically involves converting biomass 12 into sugars by hydrolysis and then fermenting the sugars to produce ethanol. Because cellulosic materials are more difficult to convert to sugar than are carbohydrates, grain is the preferred biomass used to produce ethanol.

However, ethanol facility 15 may be designed to convert virtually any biomass 12 into ethanol using techniques known in the art.

Ethanol made from a biomass 12 such as grain may be produced by a dry mill process or a wet mill process. In an embodiment of the present invention, grain biomass 12 is dry milled, although the wet mill process may be used as well. Typically in the dry mill process, after grain biomass 12 has been ground into a firm powder called meal, the meal is mixed with water and a first enzyme. The biomass mixture is then passed through cookers where it is liquefied into a mash. Heat is applied at this stage to enable liquefaction and to reduce bacteria levels in the mash. The mash is then cooled and a secondary enzyme is added to convert the mash to fermentable sugars. The biomass may also be treated with ammonia to assist in the breaking down of the biomass. The ammonia may be recovered in a process described below so as to enable the re-introduction of the ammonia to the ethanol producing process. Yeast is added to the mash to ferment the sugars to produce ethanol and carbon dioxide. The fermentation process generally takes between 40 to 50 hours. The fermented mash is then pumped to the distillation system where the ethanol is removed from the solids and the water. The solids and water are typically referred to as stillage. The ethanol is extracted from the top of a distillation column and the residual stillage is transferred from the base of the column to the anaerobic digestion facility 20.

The ethanol from the top of the column passes through a dehydration system where the remaining water may be removed. The ethanol is then denatured or made unfit for human consumption if ethanol user 35 is the fuel additive industry or industrial solvent industry. If ethanol user 35 is the natural products industry which uses ethanol for producing, as for example, extracts of various natural products such as propolis and black cohosh, the ethanol produced would not be denatured.

Ethanol producing facility 15 is similar to a conventional ethanol plant except for the absence of the drying equipment typically used to dry the whole stillage and the thin stillage to produce dried distillers grain. By eliminating such drying, handling, and storage equipment, the energy usage of ethanol producing facility 15 may be significantly reduced compared to other ethanol plants. Furthermore, there may also be a significant reduction in capital costs associated with the construction of an ethanol facility without such drying equipment. By providing anaerobic digestion facility 20 adjacent to ethanol producing facility 15, the whole and thin stillage may be transported directly to the digesters of anaerobic digestion facility 20 without drying first.

The major products of fermentation and distillation of biomass 12 include ethanol 16, carbon dioxide 17, and stillage 18. Ethanol 16 is supplied to various ethanol users 35. In one 15 embodiment of system 10, ethanol user 35 is a manufacturer of health products wherein ethanol 16 is used to prepare various herbal remedies and tinctures, vitamins, minerals and specialty supplements. In another embodiment, ethanol 16 may be sold to the fuel industry and used as a renewable fuel, primarily as a gasoline volume extender and also as an oxygenate for high-octane fuels.

Carbon dioxide 17 may be collected in storage vessels and supplied to various industries, such as manufacturers of carbonated drinks and suppliers of industrial grade carbon dioxide. In an embodiment of system 10, carbon dioxide 17 may be supplied to greenhouse 30 to facilitate photosynthesis. Carbon dioxide 17 contributes to plant growth by enabling plants to combine carbon dioxide 17 and water with the aid of light energy to form sugars which are then converted into complex compounds for continued plant growth. When the supply of carbon dioxide 17 is insufficient, plants cannot utilize the sun's energy fully and growth is inhibited.

Applicant believes that in most cases rate of plant growth under otherwise identical growing conditions is directly related to carbon dioxide concentration. Commercial growers have long used carbon dioxide to increase plant health and crop yields because increasing carbon dioxide levels accelerates photosynthesis. Plants grown in carbon dioxide enriched environments exhibit thicker, lush foliage, increased branching, and more plentifull blooms.

Stillage 18, which comprises whole stillage and thin stillage, is transported to anaerobic digestion facility 20 for anaerobic treatment. Additional organic waste 22 generated by ethanol user 35 such as residual organics from manufacturer of health products may be added to stillage 18 for anaerobic digestion. Furthermore, other organics 24 such as city waste or sewage may be provided for anaerobic digestion. Organic waste from greenhouse 30 may also be added to stillage 18 for anaerobic treatment.

Depending on the incoming waste stream (feedstock) a thermal hydrolysis (TDH) process may be used to pre-treat the organic waste before anaerobic digestion. TDH increases pressure and temperature applied to the organic part of the waste. The waste is thereby split-up in a first step into short-chain fragments that are biologically well suited for microorganisms. The following fermentation runs much faster and more complete than in conventional digestion processes and the biogas yield is increased. Left is just a small amount of a solid residue that can be easily dewatered and utilized as surrogate fuel for incineration or as compost additive. The thermal hydrolysis process allows a substantially complete energy recovery from organic waste. During the total procedure more energy sources are produced than are needed for running the plant. The procedure is especially suited for wet organic waste and biosolids that are difficult to compost, such as food scraps, biological waste from compact residential areas and sewage sludge. As a complete disinfection is granted due to the process temperatures the procedure is also suited for carcasses.

Anaerobic digestion facility 20 comprises a plurality of digesters. Digesters are large air-tight tanks which are typically made out of concrete, steel, brick, or plastic. They may be shaped like silos, troughs, basins or ponds, and may be placed underground or on the surface of the ground. A digester comprises a pre-mixing area or tank, a digester vessel, a system for collecting biogas, and a system for distributing the effluent or the remaining digested material. There are two basic types of digesters: batch and continuous. Batch-type digesters are operated by loading the digester with organic materials, allowing it to completely digest, removing the effluent, and repeating the process again. In a continuous digester, organic material is constantly fed into the digester such that biogas is continually produced without the interruption of loading organic material and unloading effluent. In an embodiment of the present invention, anaerobic digestion facility 20 comprises a plurality of continuous vertical tank digesters which are typically better suited for larger operations and produce a steady and predictable supply of usable biogas, such as methane gas 45.

Stillage 18 is transported into digesters where microorganisms convert stillage 18 into organic acids. Methane-producing (methanogenic) anaerobic bacteria utilize these acids and complete the decomposition process. The rate of digestion and biogas production depends on the temperature that the anaerobic bacteria can endure. Typically, they thrive best at temperatures of about 98° F. (36.7° C.) (mesophilic) and 130° F. (54.4° C.) (thermophilic).

Methane gas 45 and carbon dioxide 50 produced by anaerobic treatment of stillage 18 may be collected by a gas collection system and stored separately in a plurality of vessels, such as collapsible collection domes. A series of valves and tubes control the flow of gases to their respective storage locations or use locations. Methane gas 45 may be scrubbed and compressed and supplied as natural gas 47 and transported to various consumers. In an embodiment of the present invention, methane gas 45 may be transported to generator 25 where methane gas 45 is converted into electricity 48. In one embodiment, methane gas 45 is burned to heat a boiler 55. Boiler 55 produces steam to drive a turbine 60 which turns electric generators 25 to produce electricity 48 and steam. In another embodiment, methane gas 45 may be burned in turbine 60 to produce electricity 48. Electricity 48 may then be supplied to operate ethanol producing facility 15. Alternatively, electricity 48 may be sold to a local utilities company via a substation 65 to supply electricity to the city. Electricity generated from methane gas 45 is renewable and cleaner as there are no net emissions of carbon dioxide. Although the process of extracting energy from methane gas 45 is not 100% efficient, the energy lost as heat or steam 67 is collected and transported to ethanol facility 15 and used to heat the process water required to aid in the fermentation process. In embodiment an integrated recovery unit is provided which reclaims exhaust gas heat through a heat exchanger and consequently generates steam for use in the process plant.

Like carbon dioxide 17, carbon dioxide 50 produced from anaerobically digesting stillage 18 may also be supplied to greenhouse 30 to provide plants with the necessary greenhouse gas to photosynthesize. In an embodiment of the invention, greenhouse 30 may be used to grow herbs, plants and other organic products 32 to supply ethanol user 35 with the natural products to manufacture herbal remedies, tinctures, and other health products. In another embodiment of the invention, greenhouse 30 may be used to produce and supply at least some of the biomass 12 to ethanol producing facility 15.

Because anaerobic digestion is an exothermic process, hot water generated by the anaerobic digestion of stillage 18 may be used for heating greenhouse 30 to keep the plants warm enough to live in the winter. Hot water pipes may be laid near to the plants. Alternatively, hot water 70 may be supplied to heat exchangers to heat the air in greenhouse 30.

Another by-product of anaerobically treating stillage 18 is called organic slurry, or effluent. Organic slurry is rich in nutrients (ammonia, phosphorus, potassium, and more than a dozen trace elements) and is an excellent organic and pathogen free soil conditioner. In an embodiment of the present invention, the organic slurry may be provided to the plants in greenhouse 30 to facilitate their growth. Alternatively, the organic slurry may be centrifuged to 25 separate the solid from the nutrient rich water. The solid may be used as compost 75 for greenhouse 30 or dried and sold as a livestock feed additive. The remaining nutrient rich water 80 may be used as a liquid bio-fertilizer for greenhouse 30.

Alternatively, nutrient rich water 80 may be subjected to reverse osmosis to create purified water 85 to be transported to ethanol producing facility 15 and used in the production of ethanol 16. Reverse osmosis, also known as hyperfiltration, allows the removal of particles as small as ions from a solution. Reverse osmosis may be used to purify water and remove salts and other impurities in order to improve the color, taste or properties of the fluid. Reverse osmosis uses a membrane that is semi-permeable, allowing water to pass through it, while rejecting other ions that remain. As nutrient rich water 80 passes through the membrane and continues to purify nutrient rich water 80 to produce purified water 85, what remains is a liquid having an increasingly high concentration of nutrients as the membrane continually rejects the nutrients in nutrient rich water 80, thereby producing a concentrated liquid bio-fertilizer. In an embodiment of the present invention, the concentrated liquid bio-fertilizer may be provided to the plants in greenhouse 30 to facilitate their growth. In an alternative embodiment, the ammonia in the concentrated liquid bio-fertilizer may be separated out such that the ammonia may be re-introduced back into the ethanol producing process to assist in breaking down the biomass.

As will be apparent to those skilled in the art in the light of the foregoing disclosure, many alterations and modifications are possible in the practice of this invention without departing from the spirit or scope thereof. Accordingly, the scope of the invention is to be construed in accordance with the substance defined by the following claims.

What is claimed is:

1. A system of anaerobically digesting stillage and reintegrating substantially all by-products thereof back into the system, the system comprising:
a) an ethanol producing facility for producing ethanol;
b) an anaerobic digestion facility for anaerobically digesting stillage from said ethanol producing facility to produce a plurality of by-products; and
c) a plurality of sub-systems wherein said plurality of by-products are used by said plurality of sub-systems to produce a plurality of end-products; and
d) integration means for integrating at least one of said plurality of end-products back into said ethanol producing facility and into at least one of said plurality of sub-systems such that the system of anaerobically digesting stillage is a continuous and self-sustaining operation.

2. The system of claim 1 wherein said plurality of sub-systems comprise:
a) a generator sub-system for producing electricity;
b) a greenhouse sub-system for producing greenhouse end-products; and
c) an ethanol user sub-system for producing ethanol end-products.

3. The system of claim 2 wherein each of said plurality of sub-systems, said ethanol producing facility, and said anaerobic digestion facility are within close physical proximity to one another such that said plurality of sub-systems, said ethanol producing facility, and said anaerobic digestion facility, taken collectively together, form a self-contained unit.

4. The system of claim 3 wherein biomass for producing ethanol is supplied at least in part by said greenhouse sub-system, said greenhouse sub-system comprising at least one greenhouse facility.

5. The system of claim 4 wherein said greenhouse end-products comprise biomass, said biomass integrated by said integration means back into said ethanol producing for producing said ethanol.

6. The system of claim 5 wherein said biomass for producing said ethanol is grain.

7. The system of claim 6 wherein said ethanol from said ethanol producing facility is transported to said ethanol user sub-system, said ethanol user sub-system comprising a natural products manufacturer, a fuel additive manufacturer and an industrial solvent manufacturer.

8. The system of claim 7 wherein said ethanol end-products produced by said ethanol user sub-system comprises herbal remedies and tinctures, fuel oxygenate, fuel additive, and industrial solvents.

9. The system of claim 8 wherein said ethanol producing facility further produces carbon dioxide which is transported to said greenhouse sub-system such that said carbon dioxide assists photosynthesis of said greenhouse end-products.

10. The system of claim 9 wherein said greenhouse end-products further comprises a plurality of herbs and plants for said natural products manufacturer for producing said herbal remedies and tinctures with said ethanol.

11. The system of claim 10 further comprising a thermal hydrolysis facility and wherein said stillage from said ethanol producing facility is treated in said thermal hydrolysis facility and then transported to said anaerobic digestion facility, said anaerobic digestion facility substantially adjacent to said ethanol producing facility.

12. The system of claim 11 wherein any organic waste and any organic discard from said plurality of sub-systems is added to said stillage to be anaerobically digested at said anaerobic digestion facility.

13. The system of claim 12 wherein said plurality of by-products produced from said anaerobic digestion facility comprise:
a) methane gas;
b) carbon dioxide;
c) hot water; and
d) effluent.

14. The system of claim 13 wherein said anaerobic digestion facility comprises at least one air-tight digester, said digester receiving said stillage, said organic waste, and said organic discard for anaerobic digestion.

15. The system of claim 14 wherein said digester is a continuous digester wherein said stillage, said organic waste, and said organic discard are continually fed into said digester such that said methane gas and said carbon dioxide are continually produced, and said effluent and said hot water are continually removed.

16. The system of claim 15 wherein said methane gas is collected from said digester and scrubbed and compressed to be supplied as natural gas to consumers.

17. The system of claim 15 wherein said methane gas is transported from said digester to said generator sub-system to produce said electricity.

18. The system of claim 17 wherein said generator sub-system comprises a boiler and a turbine wherein said methane gas is burned to heat said boiler, said boiler producing steam to drive said turbine which turns electric generators to produce said electricity.

19. The system of claim 18 wherein said electricity is sold to a utilities company via a substation to supply electricity to end users.

20. The system of claim 18 wherein said electricity is integrated back into at least one of said plurality of sub-systems to operate said at least one sub-system.

21. The system of claim 18 wherein said electricity is integrated back into said ethanol producing facility to operate said ethanol producing facility.

22. The system of claim 21 wherein heat produced from converting said methane to said electricity is collected and transported to said ethanol producing facility, said heat used to aid in fermentation and distillation of said biomass to produce said ethanol.

23. The system of claim 22 wherein said carbon dioxide produced in said digester is collected and transported to said greenhouse sub-system to facilitate photosynthesis of said greenhouse end-products.

24. The system of claim 23 wherein said hot water from said digester is collected and used for heating said greenhouse sub-system.

25. The system of claim 24 wherein said effluent from said digester is collected for use by an end user as fertilizer and used as a soil conditioner to facilitate growth of said greenhouse end-products.

26. The system of claim 25 wherein said effluent is separated into a solid and a liquid, said solid used as compost and said liquid used as a fertilizer for said greenhouse sub-system.

27. The system of claim 26 wherein said liquid is subjected to reverse osmosis to create purified water, said purified water transported to said ethanol producing facility to produce ethanol.

28. A system of anaerobically digesting stillage and reintegrating substantially all by-products thereof back into the system, the system comprising:
a) an ethanol producing facility for producing ethanol;
b) an anaerobic digestion facility for anaerobically digesting stillage from said ethanol producing facility to produce a plurality of by-products;
c) a generator sub-system for producing electricity and/or heat from one of said plurality of by-products from said anaerobic digestion facility;
d) a greenhouse sub-system for producing a plurality of greenhouse end-products, said greenhouse sub-system integrating at least one of said plurality of by-products from said anaerobic digestion facility to facilitate growth of said plurality of greenhouse end-products; and
e) an ethanol user sub-system for producing ethanol end-products from said ethanol produced at said ethanol producing facility;
wherein interactions between said ethanol producing facility, said anaerobic digestion facility, said generator sub-system, said greenhouse sub-system, and said ethanol user sub-system integrate each of said ethanol producing facility, said anaerobic digestion facility, said generator sub-system, said greenhouse sub-system, and said ethanol user sub-system such that the system of anaerobically digesting stillage is a continuous and self-sustaining operation.

29. The system of claim 28 wherein said ethanol producing facility, said anaerobic digestion facility, said generator sub-system, said greenhouse sub-system, and said ethanol user sub-system are located within close proximity to one another such that said ethanol producing facility, said anaerobic digestion facility, said generator sub-system, said greenhouse sub-system, and said ethanol user sub-system, taken together, form a self-contained unit.

30. The system of claim 29 wherein said greenhouse sub-system comprises at least one greenhouse facility, said greenhouse facility producing, at least in part, biomass for producing said ethanol.

31. The system of claim 30 wherein said ethanol from said ethanol producing facility is transported to said ethanol user sub-system, said ethanol user sub-system comprising a natural products manufacturer, a fuel additive manufacturer and an industrial solvent manufacturer.

32. The system of claim 31 wherein said ethanol producing facility further produces carbon dioxide which is transported to said greenhouse sub-system such that said carbon dioxide may facilitate photosynthesis of said biomass.

33. The system of claim 32 wherein said stillage from said ethanol producing facility is treated in said thermal hydrolysis facility and then transported to said anaerobic digestion facility, said anaerobic digestion facility comprising at least one air tight digester for receiving said stillage.

34. The system of claim 33 wherein said plurality of by-products produced from said anaerobic digestion facility comprise:
a) methane gas;
b) carbon dioxide;
c) hot water;
d) effluent and fertilizer; and,
e) ammonia.

35. The system of claim 34 wherein said methane gas is transported from said digester to said generator sub-system to produce said electricity, said generator sub-system comprising a boiler and a turbine wherein said methane gas is burned to heat said boiler, said boiler producing steam to drive said turbine which turns electric generators to produce said electricity.

36. The system of claim 35 wherein said electricity is integrated back into said ethanol producing facility to operate said ethanol producing facility.

37. The system of claim 36 wherein heat produced from converting said methane to said electricity is collected and transported to said ethanol producing facility, said heat used to aid in fermentation of said biomass to produce said ethanol.

38. The system of claim 37 wherein said carbon dioxide produced in said digester is collected and transported to said greenhouse sub-system to facilitate photosynthesis of said greenhouse end products.

39. The system of claim 38 wherein said hot water from said digester is collected and used for heating said greenhouse sub-system.

40. The system of claim 39 wherein said effluent from said digester is collected for use as a soil conditioner.

41. The system of claim 40 wherein said effluent is separated into a solid and a liquid, said solid used as compost for said greenhouse sub-system and said liquid subjected to reverse osmosis to create purified water and a fertilizer, said purified water transported to said ethanol producing facility to produce ethanol and said fertilizer supplied to said greenhouse sub-system.

42. A method of anaerobically digesting stillage and reintegrating substantially all by-products thereof back into the system, the system comprising:
a) producing ethanol and stillage in an ethanol producing facility;
b) anaerobically digesting stillage in an anaerobic digestion facility associated with said ethanol producing facility to produce a plurality of by-products; and
c) using said plurality of by-products in a plurality of sub-systems to produce a plurality of end-products; and
d) integrating by integration means at least one of said plurality of end-products back into said ethanol producing facility and into at least one of said plurality of sub-systems such that the system of anaerobically digesting stillage is a continuous and self-sustaining operation.

43. The method of claim 42 further comprising the step of operating said plurality of sub-systems, said ethanol producing facility, and said anaerobic digestion facility within close physical proximity to one another such that said plurality of sub-systems, said ethanol producing facility, and said anaerobic digestion facility, taken collectively together, form a self-contained unit.

44. The method of claim 43 wherein said plurality of end-products includes biomass and said method further comprises the step of supplying said biomass for said step of producing ethanol, at least in part, from a greenhouse sub-system, wherein said greenhouse sub-system includes at least one greenhouse.

45. The method of claim 44 wherein said integrating step includes integrating said biomass into said ethanol producing step.

46. The method of claim 45 further comprising the step of transporting said ethanol from said ethanol producing facility to an ethanol user sub-system of said plurality of sub-systems wherein, said ethanol user sub-system includes an herbal natural products manufacturer, a fuel additive manufacturer or an industrial solvent manufacturer.

47. The method of claim 42 comprising the step of providing as part of said plurality of sub-systems:
a) a generator sub-system for producing electricity;
b) a greenhouse sub-system for producing greenhouse end-products, carbon dioxide, and fertilizer; and
c) an ethanol user sub-system for producing ethanol end-products.

48. The method of claim 44 further comprising the step of transporting carbon dioxide produced by said ethanol producing facility to said greenhouse sub-system such that said carbon dioxide may facilitate photosynthesis of said greenhouse end-products.

49. The method of claim 45 further comprising the steps of treating by thermal hydrolysis and transporting said stillage from said ethanol producing facility to said anaerobic digestion facility, and providing said anaerobic digestion facility positioned substantially adjacent to said ethanol producing facility.

50. The method of claim 45 further comprising the step of adding organic waste and organic discard from said plurality of sub-systems to said stillage to be anaerobically digested at said anaerobic digestion facility.

51. The method of claim 50 further comprising the steps of providing said anaerobic digestion facility with at least one air-tight digester, and treating by thermal hydrolysis and receiving said stillage, said organic waste, and said organic discard for anaerobic digestion in said digester.

52. The method of claim 51 wherein said digester is a continuous digester and further comprising the step of continuously feeding wherein said stillage, said organic waste, and said organic discard into said digester such that said methane gas and said carbon dioxide are continually produced, and said effluent and said hot water are continually removed.

53. The method of claim 52 further comprising the step of collecting said methane gas from said digester and scrubbing and compressing said methane gas, and supplying said natural gas for use by consumers.

54. The method of claim 52 further comprising the step of transporting said methane gas from said digester to said generator sub-system and producing said electricity.

55. The method of claim 54 further comprising the step of selling at least part of said electricity for supply of said electricity to end users.

56. The method of claim 55 further comprising the step of integrating at least part of said electricity back into at least one sub-system of said plurality of sub-systems to operate said at least one sub-system.

57. The method of claim 56 further comprising the step of collecting heat produced from conversion of said methane to said electricity and transporting said heat to said ethanol producing facility, and using said heat to aid in fermentation of said biomass to produce said ethanol.

58. The method of claim 57 wherein said carbon dioxide produced in said digester is collected and transported to said greenhouse sub-system to facilitate photosynthesis of said greenhouse end-products.

59. The method of claim 58 wherein said hot water from said digester is collected and used for heating said greenhouse sub-system.

60. The method of claim 59 wherein said effluent from said digester is collected and used as a soil conditioner to facilitate growth of said greenhouse end-products.

61. The method of claim 60 further comprising the step of separating said effluent into a solid and a liquid, and using said solid as compost and using said liquid as a fertilizer for said greenhouse sub-system.

62. The method of claim 61 further comprising the step of purifying said liquid by reverse osmosis to create purified water, and transporting said purified water to said ethanol producing facility to use in said step of producing ethanol.

63. The method of claim 44 further comprising the step of collecting carbon dioxide produced by said ethanol producing facility for further end use by a consumer.

64. The method of claim 57 further comprising the step of collecting said carbon dioxide produced in said digester for further end use by a consumer.

65. The system of claim 17 wherein said generator sub-system comprises a turbine and an integrated recovery unit wherein said methane gas is burned in said turbine so as to generate electricity and steam is generated in said integrated recovery unit.

66. The system of claim 28 further comprising an ammonia sub-system for removing ammonia from said ethanol producing facility and re-introducing said ammonia into said ethanol producing facility and said greenhouse sub-system.

67. The method of claim 47 further comprising the step of providing an ammonia sub-system for removing ammonia from said ethanol producing facility and re-introducing said ammonia into said ethanol producing facility and said greenhouse sub-system.
